(19) United States
(12) Patent Application Publication
(10) Pub. No.: US 2012/0157519 A1
(43) Pub. Date: Jun. 21, 2012

(54) TELLERIUM-CONTAINING COMPOUNDS FOR TREATING VIRAL INFECTIONS

(75) Inventors: Benjamin Sredni, Kfar Saba (IL); Michael Albeck, Ramat Gan (IL)

(73) Assignee: Biomas Ltd., Kfar Saba (IL)

(21) Appl. No.: 13/378,676

(22) PCT Filed: Jun. 16, 2010
(86) PCT No.: PCT/IL2010/000474

§ 371 Date: Mar. 5, 2012

Related U.S. Application Data
(60) Provisional application No. 61/213,507, filed on Jun. 16, 2009.
(60) Provisional application No. 61/213,920, filed on Jul. 29, 2009.

(51) Int. Cl. A61K 31/33; C07D 517/10; A61P 31/12; A61K 31/351; A61P 31/16; C07D 329/00; C07D 517/22

(52) U.S. Cl. 514/450; 514/463; 514/462; 514/452; 549/455; 549/334; 549/335; 549/348; 514/459

(57) ABSTRACT

Tellurium-containing compounds identified for use in a method of treating, or prophylaxis against, a viral infection caused by an influenza virus, uses thereof and pharmaceutical kits and pharmaceutical corn positions comprising the same, are disclosed. The tellurium-containing compounds disclosed herein are suitable for co-administration with an antiviral agent.

### FIELD AND BACKGROUND OF THE INVENTION

The present invention, in some embodiments thereof, relates to antiviral therapy and, more particularly, but not exclusively, to novel compositions and methods for treating viral infections such as those caused by influenza viruses.

Influenza viruses, of the family Orthomyxoviridae, are enveloped negative-strand RNA viruses with segmented genomes containing seven to eight gene segments. One genus includes influenza A and B viruses, and the other comprises influenza C viruses. The three virus types differ in their host range and in their pathogenicity [Taubenberger & Morens, Annu Rev Pathol 2008, 3:499-522]. Influenza A viruses, considered to be the most important human influenza pathogens, infect a wide variety of warm-blooded animals, including birds, swine, horses, and other mammals. Influenza A viruses are subdivided by antigenic characterization of the hemagglutinin (HA) and neuraminidase (NA) surface glycoproteins that project from the virion. The HA plays a key role in initiating viral infection by binding to sialic acid-containing receptors on host cells and thus mediating the subsequent viral entry and membrane fusion [Skehel & Wiley, Annu Rev Biochem 2000, 69:531-569]. Sixteen HA subtypes and 9 NA subtypes are known.

The H5N1 subtype of Influenza A includes a highly pathogenic avian virus, which is the causative agent of “avian influenza” (also referred to as “bird flu”). The differences between the human and avian cellular receptors are considered to be the main barrier for the efficient transmission of H5N1 influenza viruses between humans. A change in receptor specificity from the avian cell surface receptor SAα2,3Gal to the human cell surface receptor SAα2,6Gal is thought to be necessary before avian influenza viruses can cause a pandemic [Yamada et al., Nature 2006, 444:378-382]. Nevertheless, H5N1 avian influenza virus has been reported in poultry in 63 countries; 359 human cases have been confirmed in 14 countries, with a mortality rate of over 60% [World Health Organization].

The H1N1 subtype is the most common cause of influenza in humans. Some strains of H1N1 are endemic in humans, and other strains are endemic in swine and in birds.

On Jun. 11, 2009, the World Health Organization (WHO) raised the worldwide pandemic alert level to Phase 6 in response to the ongoing global spread of the novel influenza A (H1N1) virus of swine-origin, which causes influenza referred to as “swine influenza” or “swine flu” (a Phase 6 designation indicates that a global pandemic is underway). The swine flu virus has put the world on alert because a new flu strain (naturally hosted by pigs) has crossed the species barrier to humans, and apparently acquired the capability for human to human transmission. It is already known that the swine flu is resistance to adamantane-based drugs (e.g., adamantane and rimantadine) and sensitive to neuraminidase inhibitor such as oseltamivir (Tamiflu) and zanamivir (Relenza). One problem is that influenza viruses are subjected to genetic changes, and therefore the virus could develop a resistance to neuraminidase inhibitors as well.

Various tellurium compounds have been described in the art as having immunomodulating properties. A particularly effective family of tellurium-containing compounds is taught, for example, in U.S. Pat. Nos. 4,752,614; 4,761,490; 4,764,461 and 4,929,739, whereby another effective family is taught, for example, in PCT International Patent Application No. PCT/IL2005/000989, which are all incorporated by reference as if fully set forth herein. The immunomodulating properties of these tellurium-containing compounds are described, for example, in U.S. Pat. Nos. 4,962,207, 5,093,135, 5,102,908, 5,213,899 and in PCT International Patent Application No. PCT/IL2005/000989, which are all incorporated by reference as if fully set forth herein.

One of the most promising compounds described in these patents is ammonium trichloro(dioxyethylene-O,O′)tellurate, a non-toxic, low-molecular-weight (312 Da), synthetic organo-tellurium compound, which is also referred to herein and in the art as AS101. AS101 possesses immunomodulating properties [Rosenblatt-Bin et al., Cell Immunol 1998, 184:12-25; Sredni et al., Nature 1987, 330:173-176; Sredni et al., Nat Immun Cell Growth Regul 1988, 7:163-168; Sredni et al., Immunol Lett 1994, 43:159-165; Sredni et al., J Clin Oncol 1995, 13:2342-2353], and has shown beneficial effects in several preclinical and clinical studies. AS101 was previously demonstrated to have an inhibitory effect against mouse cytomegalovirus (MCMV) [Sredni et al., Immunol Lett 1994, 43:159-165], and human immunodeficiency virus type 1 (HIV-1) [Vonsover et al., AIDS Res Hum Retroviruses 1992, 8:613-623].

Another promising tellurium-containing compound is [TeO₄(COCH)₂]₂, which is also referred to herein and in the art as SAS.

### SUMMARY OF THE INVENTION

According to an aspect of some embodiments of the present invention there is provided a method of treating a viral infection caused by an influenza virus, the method comprising administering to a subject in need thereof a therapeutically effective amount of a tellurium-containing compound.

According to some embodiments of the invention, the administering is initiated within 72 hours of the appearance in the subject of symptoms of the viral infection.

According to an aspect of some embodiments of the present invention there is provided a method of prophylaxis against a viral infection caused by an influenza virus, the method comprising administering to a subject in need thereof a therapeutically effective amount of a tellurium-containing compound.

According to some embodiments of the invention, the method is further comprising co-administering to the subject an antiviral agent.

According to an aspect of some embodiments of the present invention there is provided a use of a tellurium-containing compound in the manufacture of a medicament for treating a viral infection caused by an influenza virus.

According to some embodiments of the invention, the medicament is for use beginning within 72 hours of the appearance of symptoms of the viral infection.

According to an aspect of some embodiments of the present invention there is provided a use of a tellurium-containing compound in the manufacture of a prophylactic medicament against a viral infection caused by an influenza virus.

According to some embodiments of the invention, the medicament is for use in combination with an antiviral agent.

According to an aspect of some embodiments of the present invention there is provided a tellurium-containing compound being identified for use in a method of treating a viral infection caused by an influenza virus.

According to some embodiments of the invention, the tellurium-containing compound is identified for use beginning within 72 hours of the appearance of symptoms of the viral infection.

According to an aspect of some embodiments of the present invention there is provided a tellurium-containing compound being identified for use in a method of prophylaxis against a viral infection caused by an influenza virus.

According to some embodiments of the invention, the tellurium-containing is being for use in combination with an antiviral agent.

According to an aspect of some embodiments of the present invention there is provided a pharmaceutical composition comprising a tellurium-containing compound and a pharmaceutically acceptable carrier, the composition being identified for use in the treatment of a viral infection caused by an influenza virus.

According to an aspect of some embodiments of the present invention there is provided a pharmaceutical composition comprising a tellurium-containing compound and a pharmaceutically acceptable carrier, the composition being identified for use in prophylaxis against a viral infection caused by an influenza virus.

According to some embodiments of the invention, the pharmaceutical is packaged in a packaging material, and identified in print, in or on the packaging material, for use in the treatment of the viral infection.

According to some embodiments of the invention, the composition is being identified for use beginning within 72 hours of the appearance of symptoms of the viral infection.

According to some embodiments of the invention, the composition is being packaged in a packaging material, and identified in print, in or on the packaging material, for use in prophylaxis against the viral infection.

According to some embodiments of the invention, the composition is being identified for use in combination with an antiviral agent.

According to some embodiments of the invention, the composition is further comprising an antiviral agent.

According to an aspect of some embodiments of the present invention there is provided a pharmaceutical composition comprising a tellurium-containing compound, an antiviral agent, and a pharmaceutically acceptable carrier.

According to an aspect of some embodiments of the present invention there is provided a pharmaceutical kit comprising a tellurium-containing compound and an antiviral agent, each being individually packaged within the kit, the kit being identified for use in treating a viral infection caused by an influenza virus.

According to some embodiments of the invention the kit is identified for use beginning within 72 hours of the appearance of symptoms of the viral infection.

According to an aspect of some embodiments of the present invention there is provided a pharmaceutical kit comprising a tellurium-containing compound and an antiviral agent, each being individually packaged within the kit, the kit being identified for use in prophylaxis against a viral infection caused by an influenza virus.

According to some embodiments of the invention, the influenza virus is an Influenza A virus.

According to some embodiments of the invention, the virus is an H5N1 virus.

According to some embodiments of the invention, the infection is an avian influenza.

According to some embodiments of the invention, the virus is an H1N1 virus.

According to some embodiments of the invention, the infection is a swine influenza.

According to some embodiments of the invention, the antiviral agent is a neuraminidase inhibitor.

According to some embodiments of the invention, the neuraminidase inhibitor is selected from the group consisting of oseltamivir, zanamivir, laninamivir and peramivir.

According to some embodiments of the invention, the neuraminidase inhibitor is oseltamivir.

According to some embodiments of the invention, the tellurium-containing compound and the antiviral agent act in synergy.

According to some embodiments of the invention, the tellurium-containing compound comprises at least one tellurium dioxo moiety.

According to some embodiments of the invention, the tellurium-containing compound has a general formula selected from the group consisting of:

a compound having general Formula I:

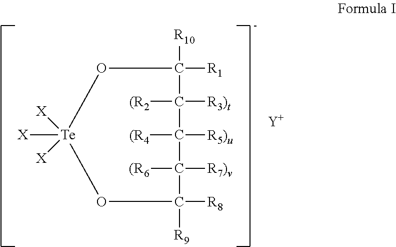

a compound having general Formula II:

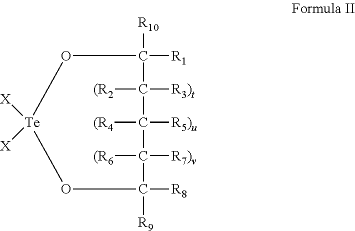

a compound having general Formula III:

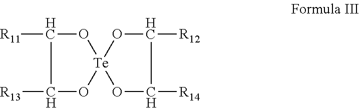

and

a compound having general Formula IV:

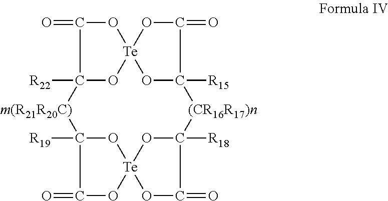

wherein:

each of t, u and v is independently 0 or 1;

each of m and n is independently 0, 1, 2 or 3;

Y is selected from the group consisting of ammonium, phsophonium, potassium, sodium and lithium;

X is a halogen atom; and

each of R₁-R₂₂ is independently selected from the group consisting of hydrogen, hydroxyalkyl, hydroxy, thiohydroxy, alkyl, alkenyl, alkynyl, alkoxy, thioalkoxy, halogen, haloalkyl, carboxy, carbonyl, alkylcarbonylalkyl, carboxyalkyl, acyl, amido, cyano, N-monoalkylamidoalkyl, N,N-dialkylamidoalkyl, cyanoalkyl, alkoxyalkyl, carbamyl, cycloalkyl, heteroalicyclic, sulfonyl, sulfinyl, sulfate, amine, aryl, heteroaryl, phosphate, phosphonate and sulfonamido.

According to some embodiments of the invention, the tellurium-containing compound has the general Formula I.

According to some embodiments of the invention, t, u and v are each 0.

According to some embodiments of the invention, each of R₁, R₈, R₉ and R₁₀ is hydrogen.

According to some embodiments of the invention, X is chloro.

According to some embodiments of the invention, Y is ammonium.

According to some embodiments of the invention, the tellurium-containing compound is ammonium trichloro(dioxoethylene-O,O′)tellurate (AS101).

According to some embodiments of the invention, the compound has the general Formula IV.

According to some embodiments of the invention, each of m and n is 0.

According to some embodiments of the invention, each of R₁₅, R₁₈, R₁₉ and R₂₂ is hydrogen.

According to some embodiments of the invention, the tellurium-containing compound is SAS.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

The term “comprising” means that other steps and ingredients that do not affect the final result can be added. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the term “pharmaceutically acceptable” means approved by a regulatory agency of the Federal or a state government or listed in the U.S. Pharmacopeia or other generally recognized pharmacopeia for use in animals, and more particularly in humans. Herein, the phrases “physiologically suitable carrier” and “pharmaceutically acceptable carrier” are interchangeably used and refer to an approved carrier or a diluent that does not cause significant irritation to an organism and does not abrogate the biological activity and properties of the administered conjugate.

As used herein, the singular form “a” “an” and “the” include plural references unless the context clearly dictates otherwise. For example, the term “a compound” or “at least one compound” may include a plurality of compounds, including mixtures thereof.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A and 1B are graphs showing the amount of H1N1 virus (FIG. 1A) and H5N1 virus (FIG. 1B) in MDCK cells pretreated with 0.1-12.8 μg/ml AS101 or without (0 μg/ml) AS101 pretreatment (results for 0 μg/ml AS101 are defined as 100%; * indicates P<0.05);

FIGS. 2A and 2B are graphs showing the amount of H1N1 virus (FIG. 1A) and H5N1 virus (FIG. 1B) in MDCK cells treated with 0.8 μg/ml AS101 24 hours before administration of the virus (pre infection), concomitantly with virus administration (with infection) or 24 hours after virus administration (post infection), or without treatment with AS101 (no treatment) (results for no treatment are defined as 100%; * indicates P<0.05);

FIG. 3 is a graph showing the body weight of mice infected with a sub-lethal dose of H5N1 virus on day 0 and treated daily with PBS, 5 μg AS101 (AS 5 ug), 10 μg AS101 (AS 10 ug) or 20 μg AS101 (AS 20 ug), and the body weight of mice not infected with the virus (control);

FIG. 4 is a graph showing the amount of H5N1 virus in infected mice which have been treated with PBS (“sick”) or AS101;

FIG. 5 is a is a graph showing the amount of H5N1 virus in infected mice which have been treated with PBS, 10 μg AS101 or 10 μg oseltamivir (Tamiflu);

FIGS. 6A and 6B are graphs showing the amount of H1N1 virus (FIG. 1A) and H5N1 virus (FIG. 1B) in lungs of mice treated with 10 μg per mouse AS101 24 hours before administration of the virus (pre infection), concomitantly with virus administration (with infection) or 24 hours after virus administration (post infection), or without treatment with AS101 (no treatment) (results for no treatment are defined as 100%; * indicates P<0.03);

FIG. 7 is a graph showing the amount of H5N1 virus (as determined by optical density in an ELISA test) in MDCK cells treated with 0.1-12.8 μg/ml AS101 in combination with 0.5-75 mg/ml oseltamivir (Tamiflu), 0.5-75 mg/ml oseltamivir alone, or without either (0 μg/ml) AS101 or oseltamivir;

FIGS. 8A and 8B are graphs showing interferon-γ (IFNγ) production (in picograms) by YTS cells (FIG. 8A) and by primary natural killer cells (FIG. 8B) in response to treatment with 0.1, 0.5, 1 or 5 μg/ml AS101, or in the absence of AS101(0 μg/ml);

FIG. 9 is a graph showing the level of γINF (in picograms) cells infected with H5N1 virus and pretreated with supernatant of untreated YTS cells (“YTS”) or YTS cells treated with 0.1, 0.5, 1, 5 or 10 μg/ml AS101, in comparison to infected MDCK cells not pretreated with supernatant (“virus”);

FIGS. 10A and 10B are graphs showing the amount of cell death of target cells caused by natural killer effector cells, as a function of the ratio of effector cells to 721.221 target cells (FIG. 10A), wherein effector cells have been pre-incubated for 24 hours with 0, 0.05, 0.1, 0.5, 1 or 5 μg/ml of AS101; results obtained with a 2.5:1 ratio of effector cells to target cells are shown as a function of AS101 concentration (FIG. 10B);

FIG. 11 is a graph showing interferon-γ (IFNγ) production (in picograms) in the lungs of uninfected mice, and in the lungs of mice infected with H5N1 virus and treated with AS101 or PBS; and

FIGS. 12A and 12B are a photograph (FIG. 12A) and plot (FIG. 12B) presenting a Western blot of phosphorylated Akt (pAkt) in MDCK cells treated with 2 μg/ml AS101 (“AS101”), 10²×TCID₅₀ of H5N1 virus (“Virus”), and 2 μg/ml AS101 in combination with 10²×TCID₅₀ of H5N1 virus (“Virus+AS101”), as well as in untreated cells (“Cells”); tubulin levels are shown (in FIG. 12A) as a control.

### DESCRIPTION OF SPECIFIC EMBODIMENTS OF THE INVENTION

The present invention, in some embodiments thereof, relates to antiviral therapy and, more particularly, but not exclusively, to novel compositions and methods for treating viral infections such as those caused by influenza viruses.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

As mentioned in the Background section hereinabove, tellurium-containing compounds have been described in the art as immunomodulators. A particularly effective family of tellurium-containing compounds is described, for example, in U.S. Pat. Nos. 4,752,614; 4,761,490; 4,764,461 and 4,929,739. The immunomodulating properties of this family of tellurium-containing compounds is described, for example, in U.S. Pat. Nos. 4,962,207, 5,093,135, 5,102,908 and 5,213,899, which are all incorporated by reference as if fully set forth herein.

One of the most promising compounds described in these patents is ammonium trichloro(dioxyethylene-O,O′)tellurate, which is also referred to herein and in the art as AS101.

As is further mentioned hereinabove, another class of tellurium-containing compounds has been disclosed in PCT/IL2005/000989. A promising tellurium-containing compound in this family is [TeO₄(COCH)₂]₂, which is also referred to herein and in the art as SAS.

The present inventors have explored the effect of tellurium-containing compounds as potent therapeutic agents against infections caused by influenza viruses.

As is demonstrated in the Examples section that follows, while reducing the present invention to practice, it was indeed found that administration of a tellurium-containing compound, either before or after infection, was efficient at inhibiting influenza virus replication and protecting mice from influenza infection, suggesting a role for tellurium-containing compounds as therapeutic and prophylactic agents against influenza viruses which are devoid of the disadvantages associated with the presently known methodologies for treating influenza viruses described hereinabove.

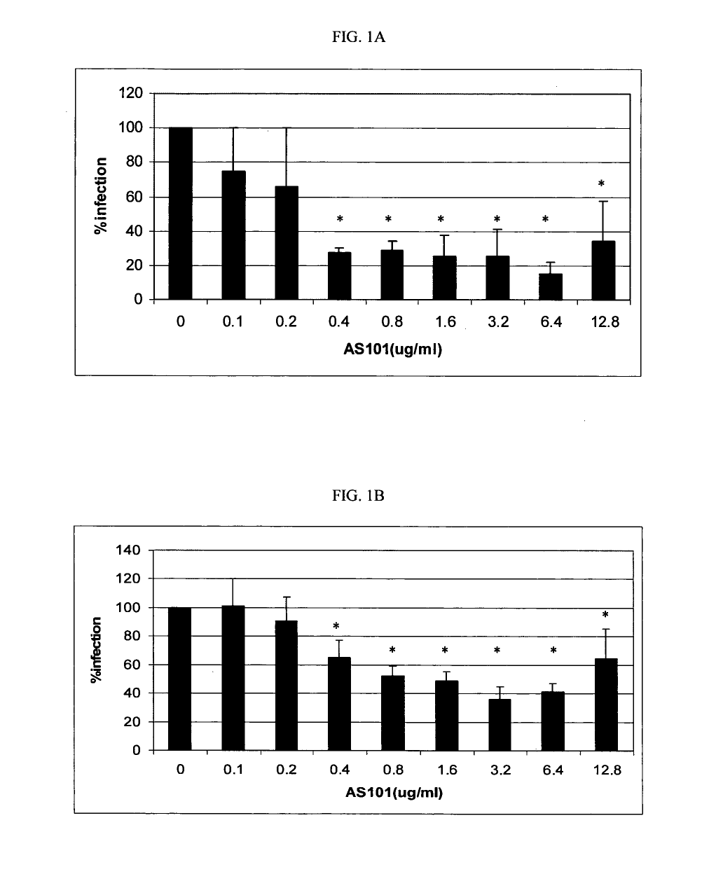

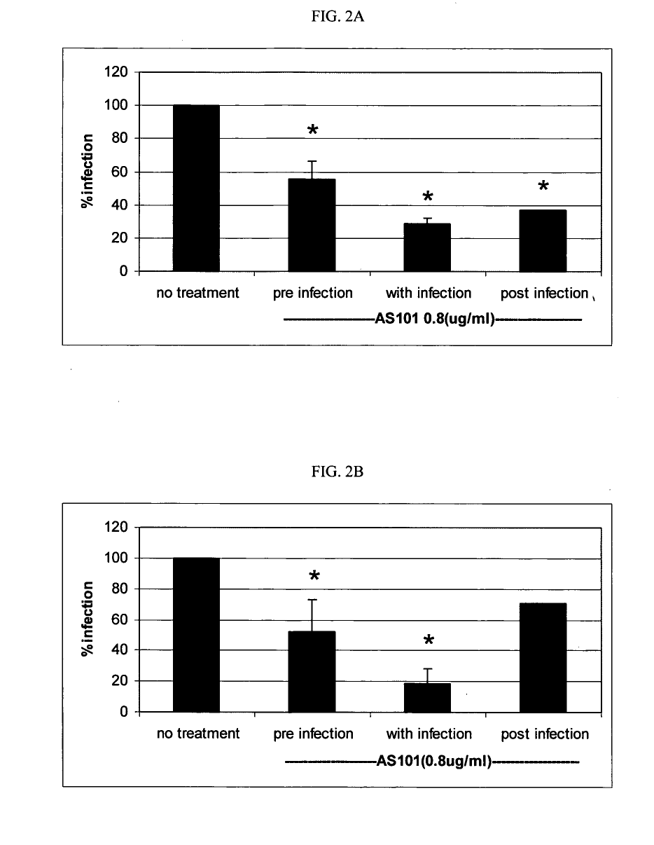

Referring now to the drawings, FIGS. 1A and 1B show inhibition of H1N1 virus (FIG. 1A) and H5N1 virus (FIG. 1B) replication in cells in vitro. FIGS. 2A and 2B show that the AS101 is effective when administered before, concomitantly with, or after infection of cells with the virus.

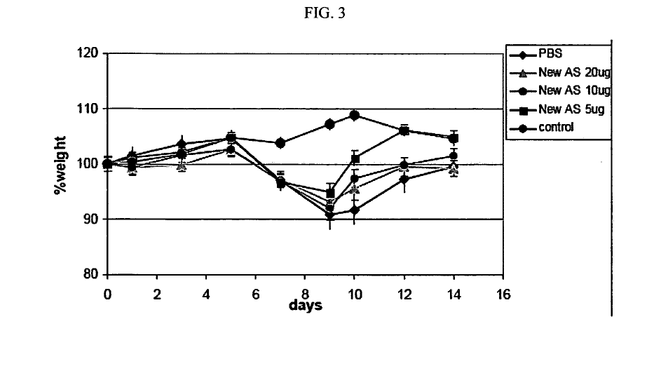

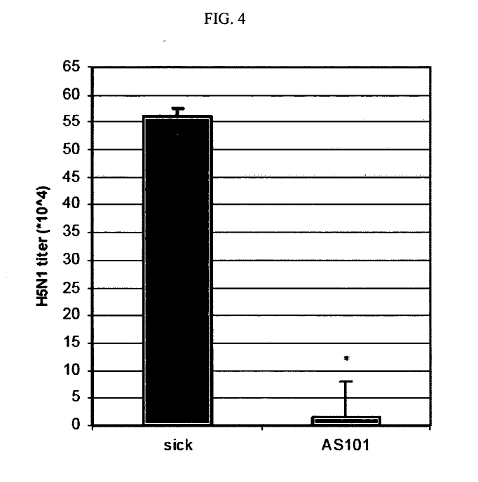

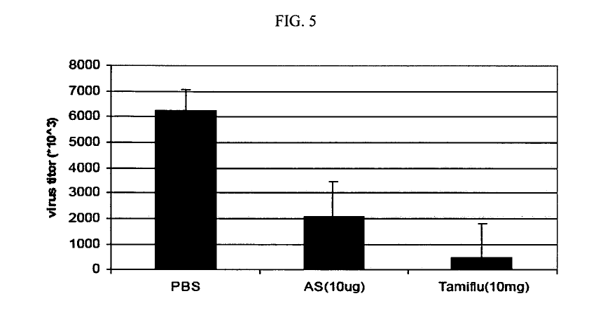

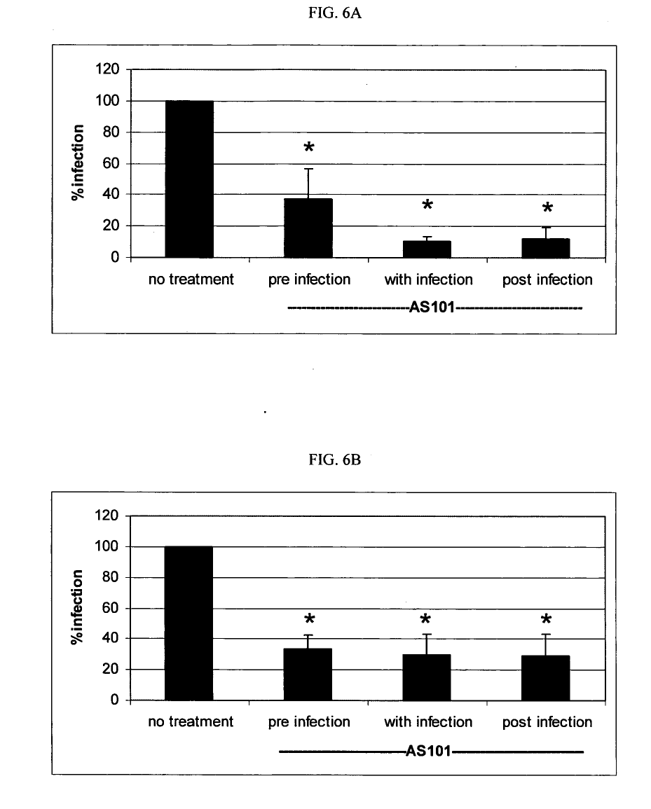

FIG. 3 shows alleviation of influenza symptoms in vivo following administration of AS101. FIGS. 4 and 5 show a considerable reduction by AS101 of the amount of H5N1 virus in vivo. FIGS. 6A and 6B show that the AS101 is effective when administered before, concomitant with, or after infection of mice with the virus.

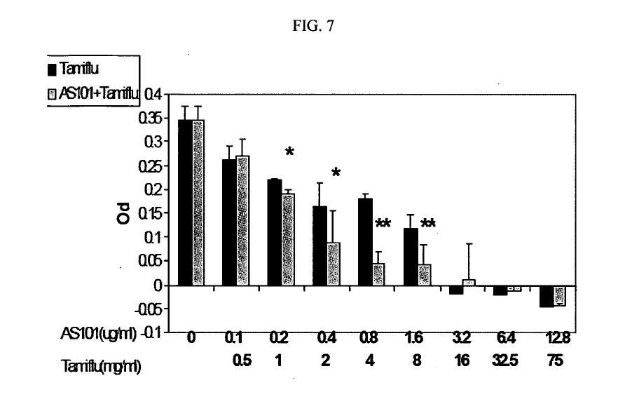

FIG. 7 shows that AS101 in combination with oselatmivir (Tamiflu) is considerably more effective at inhibiting viral replication than is oseltamivir alone, and demonstrates a synergistic activity.

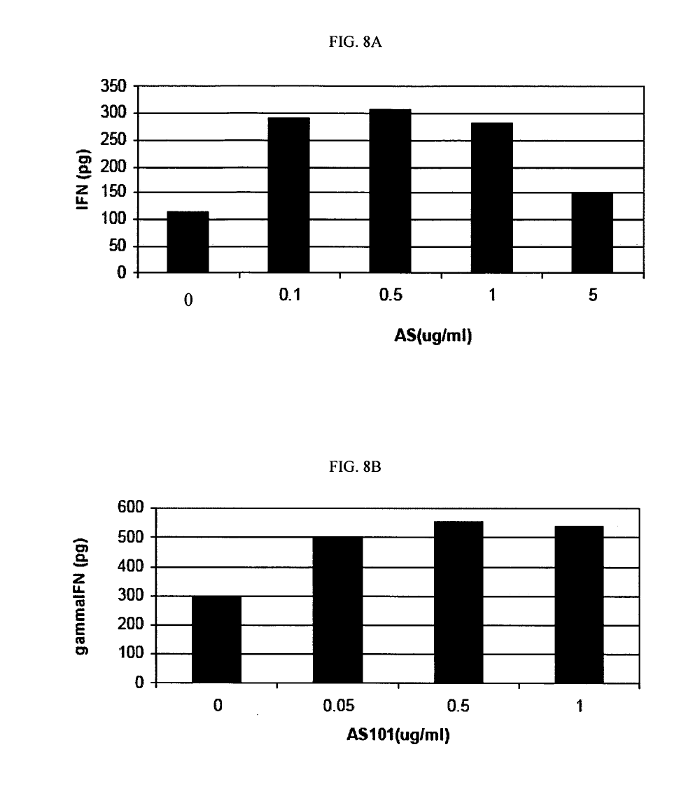

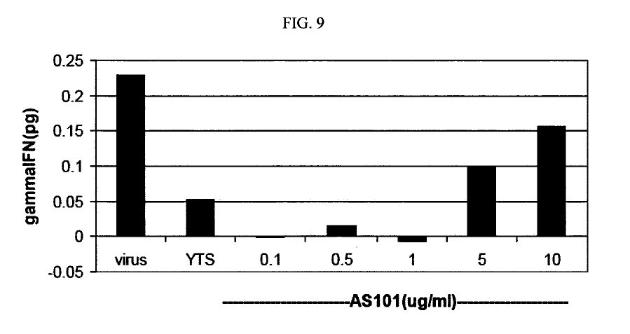

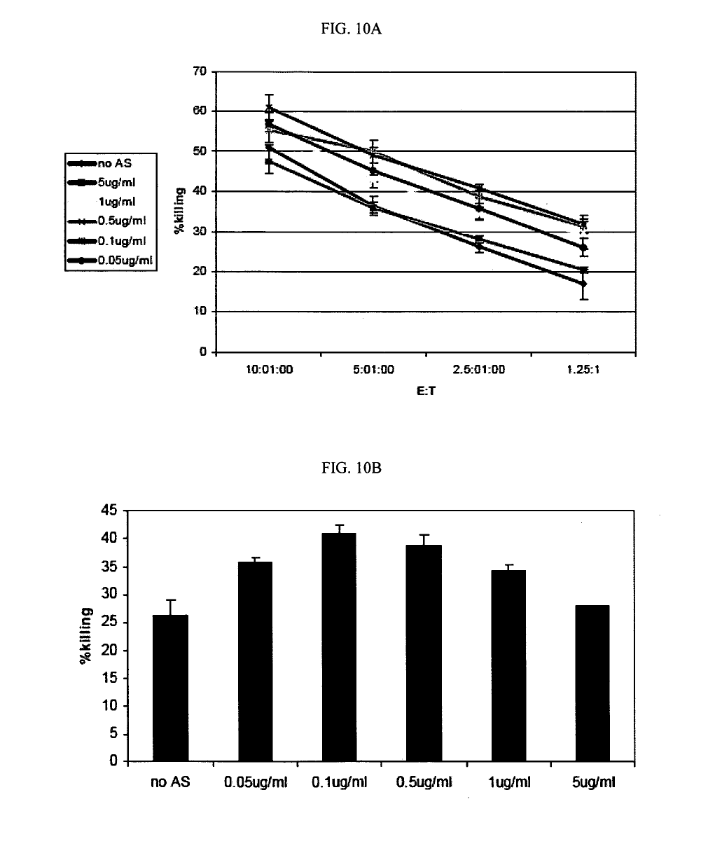

FIGS. 8A and 8B show that AS101 stimulates interferon-γ (IFNγ) production (in picograms) by YTS cells (FIG. 8A) and by primary natural killer cells (FIG. 8B) at certain doses. FIG. 9 shows that supernatant of cells treated with doses of AS101 which were found to stimulate interferon-γ production can protect cells from viral infection. FIGS. 10A and 10B show that doses of AS101 which were found to stimulate interferon-γ production enhanced cell-killing by natural killer cells.

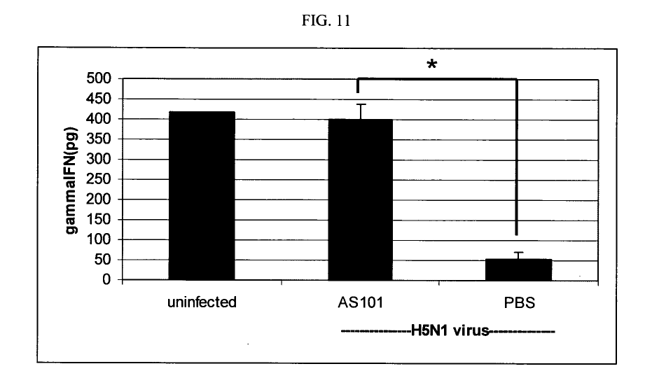

FIG. 11 shows that H5N1 virus inhibits interferon-γ production in vivo, and that AS101 restored normal interferon-γ production.

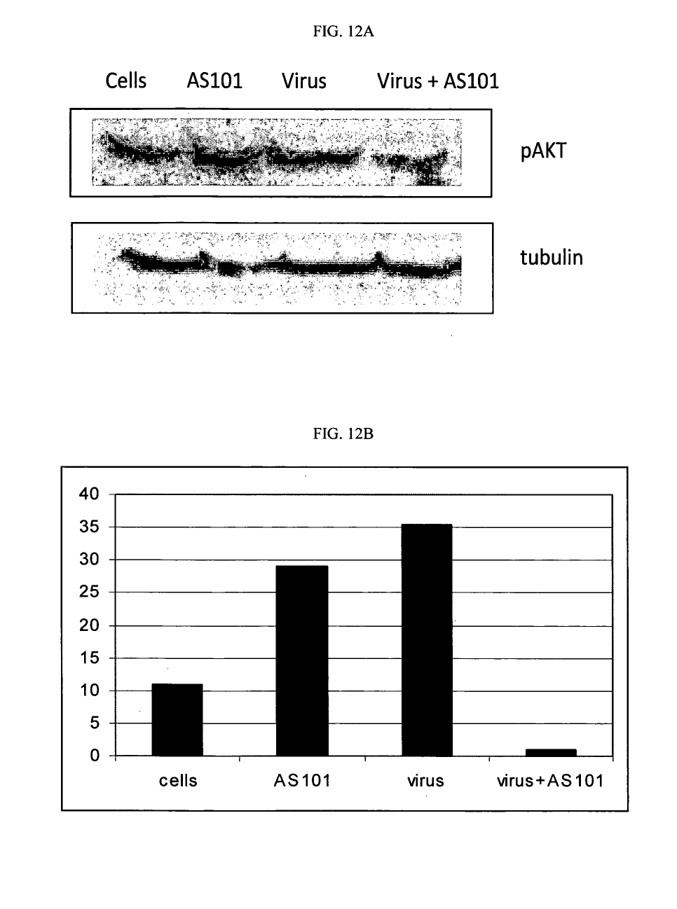

FIGS. 12A and 12B show that AS101 inhibits PI3k-Akt signaling in cells infected with influenza virus, even though both AS101 alone and influenza virus alone stimulate PI3k-Akt signaling.

These results indicate that tellurium-containing compounds can be efficiently utilized in the treatment and prevention of viral infection such as an influenza virus infection.

Thus, a novel, efficient, and convenient-to-use therapy for influenza which utilize tellurium-containing compounds is provided.

Accordingly, in one aspect of embodiments of the invention there is provided a method of treating a viral infection caused by an influenza virus. The method, according to these embodiments, is effected by administering to a subject in need thereof a therapeutically effective amount of a tellurium-containing compound.

Treating the viral infection can be effected by administering a tellurium-containing compound to the subject at the same time the subject has been infected, or, more realistically, after the subject has been infected.

Administration of the tellurium-containing compound to the subject can be initiated 1 hour, 2 hours, 3 hours, 4 hours, 6, hours, 12 hours, 24 hours, 36 hours, 48 hours, 60 hours, 72 hours and even after longer time periods, after the subject has been infected, including any value therebetween.

Since the presence of infection is typically determined only upon appearance of its symptoms in an infected subject, in some embodiments, the administration of a tellurium-containing compound is initiated upon the appearance of these symptoms.

In some cases, upon appearance of symptoms suspected as associated with a viral infection, tests are, conducted in order to confirm the presence of an Influenza virus in the subject.

Embodiments of the invention therefore encompass administration of a tellurium-containing compound which is initiated upon appearance of symptoms of the viral infection and/or upon determining the presence of an Influenza virus and/or of an infection caused by an Influenza virus by methodologies well known in the art.

Thus, in some embodiments, administering the tellurium-containing compound is initiated within 1 hour, 2 hours, 3 hours, 4 hours, 6, hours, 12 hours, 24 hours, 36 hours, 48 hours, 60 hours, 72 hours and even within longer time periods of the appearance of symptoms of the viral infection in the subject and/or upon determining the presence of an Influenza virus or of a viral infection caused by an Influenza virus in the subject.

As shown in the Examples section that follows, a tellurium-containing compound as described herein is effective in treating a viral infection even when administered 72 hours post-infection, and exhibits also a prophylactic effect when administered before the subject is infected.

Accordingly, in an aspect of embodiments of the present invention there is provided a method of prophylaxis against a viral infection caused by an influenza virus. The method, according to these embodiments, is effected by administering to a subject in need thereof a therapeutically effective amount of a tellurium-containing compound.

As used herein, the term “prophylaxis” encompasses preventing a development of a disease (e.g., a viral infection) or a development of symptoms thereof, preventing a process that can lead to a disease (e.g., a viral infection) or to a development of symptoms thereof, or preventing re-occurrence of a disease.

Such a method can be efficiently practiced in cases when a subject knows of possible exposure to the virus (e.g., due to exposure to a large population infected with the virus such as in a hospital or certain geographic area, or in certain farms) and/or of possible life-threatening risks should infection is effected.

Thus, according to some embodiments of the invention, administering a tellurium-containing compound is effected prior to a suspected exposure to an influenza virus. In some embodiments, administering the tellurium-containing compound is effected 1 hour, 2 hours, 3 hours, 4 hours, 6, hours, 12 hours, 24 hours, 36 hours, 48 hours, 60 hours, 72 hours and even longer time periods prior to a suspected exposure to an influenza virus.

Similarly, according to an aspect of some embodiments of the present invention there is provided a use of a tellurium-containing compound as described herein in the manufacture of a medicament for treating a viral infection caused by an influenza virus, as described herein.

In some embodiments, the medicament is for use beginning within a time period of the appearance of symptoms of the viral infection and/or of determining the presence of an influenza virus or an infection caused by an influenza virus in a subject.

In some embodiments, the medicament is for use beginning within 72 hours of the appearance of symptoms of the viral infection.

According to an aspect of some embodiments of the present invention there is provided a use of a tellurium-containing compound as described herein in the manufacture of a prophylactic medicament against a viral infection caused by an influenza virus, as described herein.

Further according to an aspect of some embodiments of the invention there is provided a tellurium-containing compound as described herein identified for use in a method of treating a viral infection caused by an influenza virus. In some embodiments, the tellurium-containing compound is identified for use beginning within 72 hours of the appearance of symptoms of said viral infection.

According to an aspect of some embodiments of the present invention there is provided a tellurium-containing compound as described herein, identified for use in a method of prophylaxis against a viral infection caused by an influenza virus.

In any of the methods and uses described herein for treating an infection caused by influenza virus, the administration of a tellurium-containing compound is initiated as described herein and is continued according to physician's instructions.

In any of the methods and uses described herein for prophylaxis of an infection caused by an influenza virus, administration of the tellurium-containing compound can be repeated or prolonged for any time period as desired. For example, administration of the tellurium-containing compound can be effected repeatedly as long as a suspicion of exposure to an influenza virus exists. The time interval between administration can be, for example, 12 hours, 24 hours, 48 hours, 72 hours, one week, two weeks, one month, and so forth.

In any of the methods and uses described herein, the tellurium-containing compound can be used either per se, or, preferably, as a part of a pharmaceutical composition which further comprises a pharmaceutically acceptable carrier.

Thus, according to an aspect of some embodiments of the present invention there is provided a pharmaceutical composition which comprises a tellurium-containing compound and a pharmaceutically acceptable carrier.

In some embodiments, the composition is identified for use in the treatment of a viral infection caused by an influenza virus, as described herein.

In some embodiments, the composition is identified for use in prophylaxis against a viral infection caused by an influenza virus.

In some embodiments, the composition is packaged in a packaging material, and identified in print, in or on the packaging material, for use in the treatment of the viral infection, as described herein.

In some embodiments, the composition is identified for use beginning within 72 hours of the appearance of symptoms of the viral infection and/or determining of a presence of an influenza virus or of a viral infection caused by an influenza virus in a subject.

In some embodiments, the composition is packaged in a packaging material, and identified in print, in or on the packaging material, for use in prophylaxis against the viral infection, as described herein.

As demonstrated in the Examples section that follows, it has been shown that co-administering a tellurium-containing compound as described herein enhances the therapeutic effect of the known anti-viral agent oseltamivir, also known as Tamiflu.

Moreover, indication of a synergistic effect achieved by co-administration of a tellurium-containing compound and Tamiflu can be deduced from the experiments conducted.

Accordingly, in some embodiment, in any of the methods, uses and compositions described herein, the tellurium-containing compound can be used in combination with an anti-viral agent (other than the tellurium-containing compounds described herein), e.g., by co-administering to the subject the anti-viral agent and the tellurium-containing compound. The anti-viral agent is also referred to herein as an additional anti-viral agent.

In some embodiments, the anti-viral agent is an agent capable of treating an infection caused by an influenza virus.

Examples of anti-viral agents that are suitable for use in this context of embodiments of the present invention include, but are not limited to, M2 ion channel inhibitors (e.g., amantadine and rimantadine) and neuraminidase inhibitors.

In some embodiments, the anti-viral agent is a neuraminidase inhibitor such as, but not limited to, oseltamivir, zanamivir, laninamivir and peramivir.

In some embodiments, the anti-viral agent is oseltamivir.

In any of the methods and uses described herein, the anti-viral agent can be administered to the subject prior to, concomitant with or subsequent to the administration of the tellurium-containing compound.

It is to be noted, though, that some anti-viral agent are ineffective when administered more than 24 hours from the appearance of symptoms of the infection and/or determining the presence of such an infection in the subject, while some are effective even when administered more than 3 days from the appearance of symptoms of the infection and/or determining the presence of such an infection in the subject.

Thus, depending on the anti-viral agent used, a regimen for using the tellurium-containing compound in combination with the anti-viral agent can be readily determined by a person skilled in the art (e.g., a physician).

In some embodiments, a pharmaceutical composition as described herein comprises, in addition to the tellurium-containing compound and the carrier, an anti-viral agent as described herein. Processes for co-formulating such a composition would be recognized by those skilled in the art.

Such a pharmaceutical composition can be formulated so as to release both active agents (the tellurium-containing compound and the anti-viral agent) concomitantly, or at different rates, as desired. For example, a pharmaceutical composition can be formulated so as to release at burst the tellurium-containing compound and in delayed-release form the anti-viral agent. Other formulations are also contemplated.

Alternatively, in some embodiments, a pharmaceutical composition as described herein, is identified for use in combination with an anti-viral agent as described herein. Such a composition can be a packaged composition, as described herein, which further comprises instructions (e.g., on the packaging material or within a package insert) to use in combination with an anti-viral agent, according to a recommended regimen (determined per the anti-viral agent used, as described herein).

In some embodiments, a pharmaceutical composition which comprises a tellurium-containing compound as described herein forms a part of a pharmaceutical kit which further comprises an anti-viral agent, as described herein, whereby the tellurium-containing compound and the anti-viral agent are individually packaged within the kit.

In some embodiments, the kit is identified for use in treating a viral infection caused by an influenza virus.

In some embodiments, the kit is identified for use in prophylaxis against a viral infection caused by an influenza virus.

In some embodiments, each of the tellurium-containing compound and the anti-viral agent are in a form of a pharmaceutical composition thereof, as described herein.

The kit may further comprise instructions, written on its packaging material or within a package insert, regarding the route and of administration and regimen of each of the compositions, as further discussed herein.

The infection caused by an influenza virus, which is treatable or preventable by a tellurium-containing compound as described herein, can be caused by any influenza virus, including viruses belonging to the subgenus Influenza A, Influenza B and Influenza C, and any subtype thereof.

In some embodiments the virus is an Influenza A virus, including any subtype thereof.

Exemplary subtypes of Influenza A virus include, but are not limited to, Influenza A virus subtype H1N1 (e.g., swine flu), Influenza A virus subtype H2N2 (e.g., Asian Flu), Influenza A virus subtype H3N2, Influenza A virus subtype H5N1 (e.g., Avian flue, also called Bird Flu), Influenza A virus subtype H7N7, Influenza A virus subtype H1N2, Influenza A virus subtype H9N2, Influenza A virus subtype H7N2, Influenza A virus subtype H7N3 (an avian influenza strain), Influenza A virus subtype H5N2, Influenza A virus subtype H10N7. Other subtypes of Influenza A viruses are also contemplated.

In some embodiments, the infection is caused by Influenza A virus subtype H1N1. An exemplary such infection is the viral infection currently referred to as swine flu.

In some embodiments, the infection is caused by Influenza A virus subtype H5N1. An exemplary such infection is the viral infection currently referred to as Bird flu or avian flu.

In any of the methods, compositions and uses described herein, a tellurium-containing compound, which comprises one or more tellurium atoms, is utilized.

In some embodiments, the tellurium-containing compound comprises at least one tellurium dioxo moiety.

Herein throughout, the phrases “tellurium dioxo moiety” and “tellurium dioxide moiety” are used interchangeably, and describe an —O—Te—O—, in which the tellurium center can be further substituted, or a O═Te═O.

The tellurium-containing compound may be an inorganic compound or an organic compound.

Inorganic tellurium-containing compounds include, for example, tellurium dioxide (TeO₂) per se.

Organic tellurium-containing compounds may be in the form of an organic complex such as, for example, a TeO₂ complex with citric acid or ethylene glycol, which may form TeO₂ as an end product in aqueous solutions. A representative example of the latter is the complex TeO₂HOCH₂CH₂OHNH₄Cl. Otherwise, the tellurium-containing compounds described herein include one or more tellurium atoms and one or more organic moieties that are attached thereto, for example, ammonium salts, or any other salts, of halogenated tellurium-containing compounds having a bidentate cyclic moiety attached to the tellurium atom.

Exemplary compounds in this category can be collectively represented by the general Formula I:

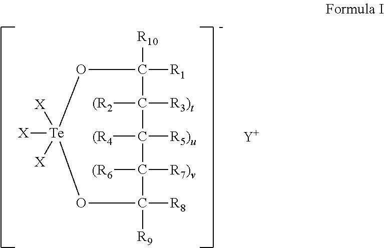

In the general Formula I above, each of t, u and v is independently 0 or 1, such that the compound may include a five-membered ring, a six-membered ring, or a seven-membered ring. In some embodiments, each of t, u and v is 0, such that the compound includes a five-membered ring.

X is a halogen atom, as described hereinabove, and is preferably chloro.

Y can be ammonium, phosphonium, potassium, sodium and lithium, and is preferably ammonium.

Each of R₁-R₁₀ is independently selected from the group consisting of hydrogen, hydroxyalkyl, hydroxy, thiohydroxy, alkyl, alkenyl, alkynyl, alkoxy, thioalkoxy, halogen, haloalkyl, carboxy, carbonyl, alkylcarbonylalkyl, alkoxy, carboxyalkyl, acyl, amido, cyano, N-monoalkylamidoalkyl, N,N-dialkylamidoalkyl, cyanoalkyl, alkoxyalkyl, carbamyl, cycloalkyl, heteroalicyclic, sulfonyl, sulfinyl, sulfate, amine, aryl, heteroaryl, phosphate, phosphonate and sulfonamido.

As used herein, the term “alkyl” refers to a saturated aliphatic hydrocarbon including straight chain and branched chain groups. In some embodiments, the alkyl group has 1 to 20 carbon atoms. In some embodiments, the alkyl is a medium size alkyl having 1 to 10 carbon atoms. In some embodiments, the alkyl is a lower alkyl having 1 to 5 carbon atoms. The alkyl group may be substituted or unsubstituted. When substituted, the substituent group can be as described herein for R₁.

As used herein, the term “hydroxyalkyl” refers to an alkyl, as this term is defined herein, substituted by a hydroxy group, as defined herein, and includes, for example, hydroxymethyl, hydroxyethyl, hydroxypropyl and hydroxy-n-butyl.

As used herein, the term “halogen”, which is also referred to herein interchangeably as “a halogen atom” or “halo”, includes chloro (Cl), bromo (Br), iodo (I) and fluoro (F).

The term “haloalkyl” refers to an alkyl, as this term is defined herein, substituted by a halogen, as defined herein, and includes, for example, chloromethyl, 2-iodoethyl, 4-bromo-n-butyl, iodoethyl, 4-bromo-n-pentyl and the like.

The term “alkanoyloxy” refers to a carbonyl group, as define herein and includes, for example, acetyl, propionyl, butanoyl and the like.

The term “carboxyalkyl” refers to an alkyl, as this term is defined herein, substituted by a carboxy group, as defined herein, and includes, for example, carboxymethyl, carboxyethyl, ethylenecarboxy and the like.

The term “alkylcarbonylalkyl” refers to an alkyl, as this term is defined herein, substituted by a carbonyl group, as defined herein, and includes, for example, methanoylmethyl, ethanoylethyl and the like.

The term “amidoalkyl” refers to an alkyl, as this term is defined herein, substituted by an amide group, as defined herein, and includes, for example, —CH₂CONH₂; —CH₂CH₂CONH₂; —CH₂CH₂CH₂CONH₂ and the like.

The term “cyanoalkyl” refers to an alkyl, as this term is defined herein, substituted by an cyano group, as defined herein, and includes, for example, —CH₂CN; —CH₂CH₂CN; —CH₂CH₂CH₂CN and the like.

The term “N-monoalkylamidoalkyl” refers to an alkyl, as this term is defined herein, substituted by an amide group, as defined herein, in which one of R′ and R″ is an alkyl, and includes, for example, —CH₂CH₂CONHCH₃, and —CH—₂CONHCH₂CH₃.

The term N,N-dialkylamidoalkyl refers to an alkyl, as this term is defined herein, substituted by an amide group, as defined herein, in which both R′ and R″ are alkyl, and includes, for example, —CH₂CON(CH₃)₂; CH₂CH₂CON(CH₂—CH₃)₂ and the like.

A “cycloalkyl” group refers to an all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or unsubstituted. When substituted, the substituent group can be as described herein for R1.

An “alkenyl” group refers to an alkyl group which consists of at least two carbon atoms and at least one carbon-carbon double bond.

An “alkynyl” group refers to an alkyl group which consists of at least two carbon atoms and at least one carbon-carbon triple bond.

An “aryl” group refers to an all-carbon monocyclic or fused-ring polycyclic (i.e., rings which share adjacent pairs of carbon atoms) groups having a completely conjugated pi-electron system. Examples, without limitation, of aryl groups are phenyl, naphthalenyl and anthracenyl. The aryl group may be substituted or unsubstituted. When substituted, the substituent group can be as described herein for R1.

A “heteroaryl” group refers to a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or unsubstituted. When substituted, the substituent group can be as described herein for R1.

A “heteroalicyclic” group refers to a monocyclic or fused ring group having in the ring(s) one or more atoms such as nitrogen, oxygen and sulfur. The rings may also have one or more double bonds. However, the rings do not have a completely conjugated pi-electron system. Examples, without limitation, include, piperazine, piperidine, morpholine, tetrahydrofuran and tetrahydropyran. The heteroalicyclic may be substituted or unsubstituted. When substituted, the substituent group can be as described herein for R1.

A “hydroxy” group refers to an —OH group.

An “alkoxy” group refers to both an —O-alkyl and an —O-cycloalkyl group, as defined herein.

An “aryloxy” group refers to both an —O-aryl and an —O-heteroaryl group, as defined herein.

A “thiohydroxy” group refers to a —SH group.

A “thioalkoxy” group refers to both an —S-alkyl group, and an —S-cycloalkyl group, as defined herein.

A “thioaryloxy” group refers to both an —S-aryl and an —S-heteroaryl group, as defined herein.

A “carbonyl” group refers to a —C(═O)—R′ group, where R′ is hydrogen, alkyl, alkenyl, cycloalkyl, aryl, heteroaryl (bonded through a ring carbon) or heteroalicyclic (bonded through a ring carbon) as defined herein.

A “thiocarbonyl” group refers to a —C(═S)—R′ group, where R′ is as defined herein.

A “carboxy” group refers to a —C(═O)—O—R′ or a —O—C(═O)—R′ group, where R′ is as defined herein.

A “sulfinyl” group refers to an —S(═O)—R′ group, where R′ is as defined herein.

A “sulfonyl” group refers to an —S(═O)₂—R′ group, where R′ is as defined herein.

A “sulfate” group refers to a —O—S(═O)₂—OR′ group, where R′ is as defined herein.

A “sulfonamido” group refers to a —S(═O)₂—NR′R″ group or a R′S(═O)₂—NR″, with R′ is as defined herein and R″ is as defined for R′.

A “carbamyl” or “carbamate” group refers to an —OC(═O)—NR′R″ group or a R″OC(═O)—NR′— group, where R′ and R″ are as defined herein.

A “thiocarbamyl” or “thiocarbamate” group refers to an —OC(═S)—NR′R″ group or an R″OC(═S)NR′— group, where R′ and R″ are as defined herein.

An “amino” group refers to an —NR′R″ group where R′ and R″ are as defined herein.

An “amido” group refers to a —C(═O)—NR′R″ group or a R′C(═O)—NR″ group, where R′ and R″ are as defined herein.

A “nitro” group refers to an —NO₂ group.

A “cyano” group refers to a —C≡N group.

The term “phosphonyl” describes a —O—P(═O)(OR′)(OR″) group, with R′ and R″ as defined hereinabove.

The term “phosphinyl” describes a —PR′R″ group, with R′ and R″ as defined hereinabove.

As cited hereinabove, the compounds in this category are salts of organic tellurium-containing compounds. The salts can be, for example, ammonium salts, phosphonium salts and alkaline salts such as potassium salts, sodium salts, lithium salts and the like.

Hence, Y in Formula I above can be a phosphonium group, as defined herein, an ammonium group, as defined herein, potassium (K⁺), sodium (Na⁺) or lithium (Li⁺).

As used herein, the term “phosphonium” describes a —P⁺R′R″R′″ group, with R′ and R″ as defined herein and R′″ is as defined for R′. The term “phosphonium”, as used herein, further refers to a —P⁺R₆ group, wherein each of the six R substituents is independently as defined herein for R, R″ and R′″.

The term “ammonium” describes a —N⁺R′R″R′″ group, with R′, R″ and R′″ as defined herein.

In some embodiments, compounds in this category include compounds having the general Formula I described above, in which Y is ammonium or phosphonium, t, u and v are each 0, and each of R₁, R₈, R₉ and R₁₀ is independently hydrogen or alkyl. These compounds can be represented by the following structure:

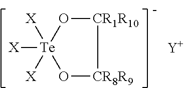

wherein each of R₁, R₈, R₉ and R₁₀ is independently hydrogen or alkyl, whereas, in some embodiment, the alkyl is methyl, and X is halogen, preferably chloro.

In some embodiments, a tellurium-containing compound for use in the context of the present embodiments has the following structure:

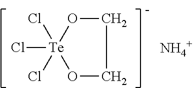

This compound is ammonium trichloro(dioxyethylene-O,O′)tellurate, which is also referred to herein and in the art as AS101.

Additional representative examples of organic tellurium-containing compound that are suitable for use in the context of the present invention include halogenated tellurium having a bidentate cyclic moiety attached to the tellurium atom. The bidentate cyclic moiety is preferably a dioxo ligand having two oxygen atoms attached to the tellurium atom.

Exemplary compounds in this category can be represented by the general Formula II:

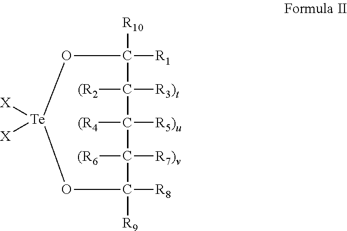

wherein t, u, v, X and R₁-R₁₀ are as defined hereinabove.

In some embodiments, the tellurium-containing compounds are those in which t, u, and v are each 0, and X is chloro, such as, but not limited to, the compound having the following structure:

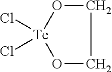

The above compound is also known in the art and referred to herein as AS103.

The organic tellurium-containing compounds having Formulae I and II can be readily prepared by reacting tetrahalotelluride such as TeCl₄ with a dihydroxy compound, as is described in detail in U.S. Pat. Nos. 4,752,614, 4,761,490, 4,764,461 and 4,929,739, which are incorporated by reference as if fully set forth herein.

Additional representative examples of organic tellurium-containing compounds that are suitable for use in the context of the present embodiments include compounds in which two bidentatic cyclic moieties are attached to the tellurium atom. Preferably, each of the cyclic moieties is a dioxo moiety.

Exemplary compounds in this category are collectively represented by the general Formula III:

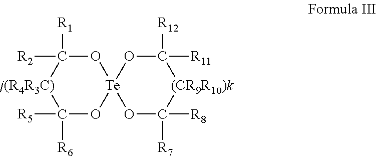

In the general Formula III above, each of j and k is independently an integer from 0 to 4, such that the compound may include a five-membered ring, a six-membered ring, a seven-membered ring, an eight-membered ring and/or a nine-membered ring. In some embodiments, each of j and k is an integer from 0 to 2, such that the compound includes a five-membered ring, a six-membered ring and/or a seven-membered ring. In some embodiments, each of j and k is 0.

R₁-R₁₂ are as defined hereinabove for R₁-R₁₀.

In some embodiments, tellurium-containing compounds in this category are those in which j and k are each 0, and R₃, R₄, R₉ and R₁₀ are each hydrogen, having the following structure:

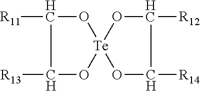

wherein each of R₁₁-R₁₄ is independently selected from the group consisting of hydrogen, hydroxyalkyl, hydroxy, thiohydroxy, alkyl, alkenyl, alkynyl, alkoxy, thioalkoxy, halogen, haloalkyl, carboxy, carbonyl, alkylcarbonylalkyl, alkoxy, carboxyalkyl, acyl, amido, cyano, N-monoalkylamidoalkyl, N,N-dialkylamidoalkyl, cyanoalkyl, alkoxyalkyl, carbamyl, cycloalkyl, heteroalicyclic, sulfonyl, sulfinyl, sulfate, amine, aryl, heteroaryl, phosphate, phosphonate and sulfonamido, as these terms are defined herein.

In some embodiments, a tellurium-containing compound in this category is a compound in which each of R₁₁-R₁₄ is hydrogen. This compound is also known in the art and referred to herein as AS102.

Additional representative examples of organic tellurium-containing compounds that are suitable for use in the context of the present embodiments include the recently disclosed ditellurium compounds having general Formula IV:

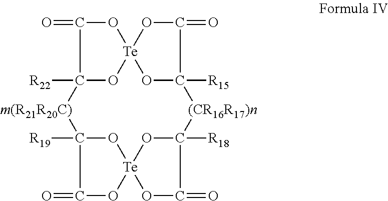

wherein each of R₁₅-R₂₂ is independently selected from the group consisting of hydrogen, hydroxyalkyl, hydroxy, thiohydroxy, alkyl, alkenyl, alkynyl, alkoxy, thioalkoxy, halogen, haloalkyl, carboxy, carbonyl, alkylcarbonylalkyl, alkoxy, carboxyalkyl, acyl, amido, cyano, N-monoalkylamidoalkyl, N,N-dialkylamidoalkyl, cyanoalkyl, alkoxyalkyl; carbamyl, cycloalkyl, heteroalicyclic, sulfonyl, sulfinyl, sulfate, amine, aryl, heteroaryl, phosphate, phosphonate and sulfonamide, as these terms are defined herein; and

m and n are each an integer from 0 to 3.

Exemplary compounds in this category are those in which m and n are each 0.

An exemplary compound in this family is a compound in which R₁₅, R₁₈, R₁₉ and R₂₂ are all hydrogen, referred to hereinafter as SAS, and which has the following structure:

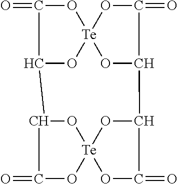

According to some embodiments of the present invention, the tellurium-containing compound is either AS101 or SAS, as described herein.

The compounds described above can be administered or otherwise utilized in the various aspects of the present invention, either as is or as a pharmaceutically acceptable salt thereof.

The phrase “pharmaceutically acceptable salt” refers to a charged species of the parent compound and its counter ion, which is typically used to modify the solubility characteristics of the parent compound and/or to reduce any significant irritation to an organism by the parent compound, while not abrogating the biological activity and properties of the administered compound.

In any of the methods and uses described herein, administration of the tellurium-containing compound and optionally of additional active agents (e.g., an anti-viral agent) can be performed via various routes of administrations.

Suitable routes of administration may, for example, include the inhalation, oral, buccal, rectal, transmucosal, transdermal, intradermal, transnasal, intestinal and/or parenteral routes; the intramuscular, subcutaneous and/or intramedullary injection routes; the intrathecal, direct intraventricular, intravenous, intraperitoneal, intranasal, and/or intraocular injection routes; and/or the route of direct injection into a tissue region of a subject.

The methods, compositions and uses described herein utilize the tellurium-containing compound and any of the optional additional active agents (e.g., an anti-viral agent) in a therapeutically or prophilactically effective amount.

Determination of a therapeutically effective amount and of a prophilactically effective amount of a tellurium-containing compound and any other active agent is well within the capability of those skilled in the art.

For any preparation used in the methods and uses of the invention, the a therapeutically or prophilactically effective amount or dose can be estimated initially from in vitro assays. For example, a dose can be formulated in animal models and such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. [See e.g., Fingl, et al., (1975) “The Pharmacological Basis of Therapeutics”, Ch. 1 p. 1].

Dosing can be of a single administration or a plurality of administrations, as described herein.

When administering systemically, a therapeutically effective amount of the tellurium-containing compounds described herein may range, for example, from about 0.01 mg/m²/day to about 20 mg/m²/day and thus can be for example, 0.01 mg/m²/day 0.02 mg/m²/day, 0.03 mg/m²/day, 0.04 mg/m²/day, 0.05 mg/m²/day, 0.1 mg/m²/day, 0.5 mg/m²/day, 1 mg/m²/day, 2 mg/m²/day, 3 mg/m²/day, 4 mg/m²/day, 5 mg/m²/day, and up to 10 mg/m²/day.

When administered orally in humans, a daily dose typically ranges between 0.1 mg and 200 mg, more preferably between 1 mg and 100 mg, depending on the age and weight of the subject. The total daily dose may be administered as a single dosage, or may be divided into a number of separate doses.

Pharmaceutical compositions comprising one or more tellurium-containing compounds as described herein may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with embodiments of the present invention may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients may be formulated in aqueous solutions, preferably in physiologically compatible buffers such as Hank's solution, Ringer's solution, or physiological salt buffer.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

A preparation according to embodiments of the present invention may also be formulated in rectal compositions such as suppositories or retention enemas, using, e.g., conventional suppository bases such as cocoa butter or other glycerides.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise glass, plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

In one embodiment, a concentration of tellurium-containing compound in the carrier ranges from about 0.01 weight percent to about 50 weight percents, more preferably from about 0.1 weight percent to about 25 weight percents, of the total weight of the composition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Methods

Materials:

AS101 was synthesized as described in Sredni et al. [Nature 1987, 330:173-176].

o-Phenylenediamine was obtained from Sigma.

Strain specific (anti-H5N1 and anti-H1N1) anti-influenza antibodies were obtained from the World Health Organization.

Peroxidase-conjugated goat anti-mouse IgG antibodies (catalog #80351) were obtained from Jackson ImmunoResearch.

Anti-pAkt mouse antibody was obtained from Santa Cruz Biotechnology.

Viruses:

Avian influenza strain A/VNH5N1—RG A/Viet Nam/1194/2004 was grown in the allantoic cavity of 11-day-old embryonated hen eggs.

The swine influenza H1N1v virus used was isolated from an Israeli patient in 2009 and was grown in MDCK cell cultures. The virus was identified using real-time PCR with a specific probe against swine influenza virus hemagglutinin (HA).

Virus growth and purification were performed according to standard methods as described by Barret and Inglis [Growth Purification and Titration Influenza Viruses in Virology: A Practical Approach 1985, IRL Press, Washington, D.C.]. The titer of virus used for infection was evaluated by the infection of Madin-Darby canine kidney (MDCK) cells, as described by Levi et al. [J Virol Methods 1995, 52:55-64], and hence, virus titer was expressed as the tissue culture infective doses leading to 50% infected cells (TCID₅₀). H5N1 influenza stock titers of 3×10⁵ TCID₅₀/ml and H1N1 influenza stock titers of 3×10⁶TCID₅₀/ml were used.

Mice:

BALB/c mice (6 weeks old) were purchased from Harlan Laboratories (Rehovot, Israel).

In Vitro Cell Infection Assay:

MDCK cells were plated in 96-well plates 3 hours before virus infection at 10⁴ cells per well in 100 μl of DMEM (Dulbecco's modified Eagle medium) supplemented with 2% fetal calf serum. Influenza strains were diluted in DMEM and were then added to the wells. H5N1 stock titer was diluted 1:3000 and H1N1 stock titer was diluted 1:5. The plates were further incubated at 37° C. for 72 hours. The cells were washed three times with PBS-T (PBS containing 0.05% (v/v) Tween 20) and 100 units of strain-specific anti-influenza antibody was added to each well. The plates were then incubated for 1 hour at room temperature. After washing three times with PBS-T, 100 units of peroxidase-conjugated goat anti-mouse IgG antibodies were added to each well, and the plates were incubated for 1 hour at room temperature. Finally, 100 μl of o-phenylenediamine (OPD) was added to each well after washing five times with PBS-T. The absorption at 492 nm was measured for each plate using a multichannel ELISA reader (Titertek, Multiskan MCC/340 MK II; Helsinki, Finland).

In Vivo Mouse Infection Assay:

BALB/c mice were inoculated intranasally with a sub-lethal dose of influenza virus in allantoic fluid containing 10² TCID₅₀. This amount of virus is equivalent to 0.4 LD₅₀, as was determined in calibration experiments.

AS101 dissolved in PBS was administrated to mice daily by intraperitoneal injection at a concentration of 10 μg per mouse (except when stated otherwise).

Mice were sacrificed 5 or 6 days after virus inoculation (except when stated otherwise), which was determined to be the time point associated with peak virus titer. To determine the virus titer, lungs were homogenized in DMEM using a homogenizer (Omni), RNA was extracted from the lungs homogenates, and the virus titer was measured by a real-time RT-PCR assay, as described by Hindiyeh et al. [J Clin Microbiol 2005, 43:589-595].

Real-Time PCR Assay:

An ABI PRISM® 7700 sequence detection system (Applied Biosystems, Foster City, Calif.) was used for amplification and detection of influenza virus A and B amplicons using TaqMan chemistry. The primers and probes specific for influenza viruses A and B used in this study were previously described by Hindiyeh et al. [J Clin Microbiol 2005, 43:589-595].

Interferon-γ (IFNγ) Assay:

A DuoSet® ELISA development kit (R&D Systems, catalog #DY285) was used in order to measure IFNγ levels according to the manufacturers instructions. Briefly, 96-well plates were coated with capture antibodies for 24 hours, and then washed with PBS-Tween. Biotinylated goat anti-mouse IFNγ was used as detection antibody, and strepavidin-horseradish peroxidase (HRP) and 3,3′,5,5′-tetramethylbenzidine (TMB) were added after incubation. IFNγ levels were measured by measuring absorption at 450 nm using an ELISA reader (Titertek) and compared to standard recombinant IFNγ.

Natural Killer (NK) Cell Killing Assay:

NK cells were incubated with AS101 for 24 hours and then incubated with 721.221 target cells. Radioactive measurement of the cell medium indicated the amount of target cell death caused by incubation with NK effector cells.

Determination of pAKT by Western Blot Analysis:

MDCK cells were incubated with 2 μg/ml AS101 and infected with 10²×TCID₅₀ of H5N1 virus. After 24 hours of incubation at 37° C., cells were lysed with ice-cold RIPA lysis buffer, proteins were loaded on polyacrilamide gel and pAKT levels were measured by Western blot analysis using anti-pAKT mouse antibody for detecting phosphorylated Ser⁴⁷³. Tubulin was measured as a loading control. Procedures were as described in Li et al. [Nature 1998, 396:580-584].

Statistical Analysis:

Statistical analysis of the differences between treated cells and controls was assessed with an unpaired Student's t-test. Statistical significance was established at a value of P<0.05.

### Results

In Vitro Cell Infection Assay:

The effect of AS101 on H5N1 (avian) and H1N1 (swine) influenza infection was investigated in vitro in MDCK cells, as described hereinabove. MDCK cells were treated with AS101 24 hours before being infected with a virus, and viral titers were measured by ELISA 72 hours post-infection.

As shown in FIGS. 1A and 1B, treatment with at least 0.4 μg/ml AS101 treatment resulted in significant inhibition of replication of both the H1N1 (FIG. 1A) and the H5N1 (FIG. 1B) influenza virus.

At the most effective concentrations AS101 was not toxic to the cells, as determined by MTT (dimethyl thiazolyl diphenyl tetrazolium) assay (data not shown).

To further examine the effect of AS101 on infection, 0.8 μg/ml AS101 was added to MDCK cells either 24 hours before the infection by the virus, concomitantly with the virus, or 24 hours following infection, and viral titers were analyzed by ELISA 72 hours post-infection.

As shown in FIGS. 2A and 2B, inhibition of both H1N1 (FIG. 2A) and H5N1 (FIG. 2B) virus infection was observed for each of the tested AS101 administration times. The strongest inhibition was observed when AS101 was administered concomitantly with the virus.

These results suggest that AS101 may be used as a prophylactic measure and as treatment against influenza viruses.

In Vivo Mouse Infection Assay:

The effect of AS101 on H5N1 (avian) and H1N1 (swine) influenza infection was further investigated in vivo in mice, as described hereinabove.

Mice were divided into five groups of six mice; one group was not infected (control group), the second one was infected without AS101 treatment and the other three groups received different concentrations of AS101. Each mouse was infected intranasally with a sub-lethal dose of H5N1 influenza virus. AS101 diluted with PBS was injected intraperitoneally every day starting from day −1 until day 12. Body weight was monitored during these 12 days after viral infection.

As can be seen in FIG. 3, all groups lost approximately 10% of their body weight relative to the control at the peak of infection, but that weight loss was reduced somewhat by each of the tested doses of AS101, indicating protection from infection by AS101.

In addition, mice were infected with H5N1 or H1N1 virus by intranasal administration, and the effect of AS101 treatment 24 hours before infection was determined as described hereinabove.

As shown in FIG. 4, significant inhibition of H5N1 virus was detected in the AS101-treated mice, as compared to control (PBS-treated) mice.

Similarly, as shown in FIG. 5, significant inhibition of the H1N1 virus was detected in the AS101-treated mice, as compared to control (PBS-treated) mice. Inhibition by 10 μg oseltamivir (Tamiflu) is shown for comparison.

In a further experiment, AS101 was administered to healthy mice 24 hours before infection, concomitantly with infection, or 24 hours following infection with sub-lethal doses of H1N1 swine influenza virus (16×TCID₅₀) or H5N1 avian influenza virus (1.6×TCID₅₀).

As shown in FIGS. 6A and 6B, AS101 treatment significantly reduced the virus titers observed in the lungs of treated mice for each of the tested AS101 administration times.

These results are in agreement with the in vitro data and demonstrate the potential of AS101 as an anti-influenza drug.

In Vitro Infection Assay Showing Effect of AS101 with Oseltamivir:

Oseltamivir (Tamiflu), is an antiviral drug used in the treatment of both Influenza virus A and Influenza virus B infections. Significantly, all recent H1 viruses that have emerged in the population worldwide have a mutation in the protein which prevents oseltamivir activity.

The effect of combined treatment with both AS101 and oseltamivir was therefore tested, using MDCK cells as described hereinabove. MDCK cells were incubated with AS101 and oseltamivir 24 hours before infection by H5N1.

As shown in FIG. 7, the combined treatment of AS101+oseltamivir was significantly more effective than oseltamivir alone in inhibiting the H5N1 virus.

These results indicate that co-administration of AS101 can effectively enhance the efficacy of oseltamivir.

In Vitro Interferon-γ (IFNγ) Assay:

To study the mechanism of action of AS101, further studies concentrated on the activity of NK (natural killer) cells, which are known as the first line of defence against pathogens [Biron et al., Annu Rev Immunol 1999, 17:189-220]. In addition, Ho et al. [J Virol 2008, 82:2028-2032] showed that infection with H5N1 virus enhances lysis of infected cells by primary human NK lines. In response to virus infection, NK cells produce IFN-γ which also depends on the presence of T cells and IL-2 [He et al., J Clin Investig 2004, 114:1812-1819]. Because AS101 was also shown to affect T cell responses [Shohat et al., Acta Derm Venereol 2001, 81:255-257], the effect of AS101 on the response of NK cells to H5N1 infection was tested.

NK line cells (YTS cells) and primary NK cells were incubated with increasing doses of AS101 for 24 hours. Cells were then centrifuged and IFN-γ levels in the supernatant were measured using ELISA assay, as described hereinabove.

As shown in FIGS. 8A and 8B, both types of NK cells exhibited increased secretion of IFN-γ following AS101 treatment in a dose dependant manner. In YTS cells, the effect on IFN-γ secretion decreased at AS101 concentrations above 1 μg/ml.

The effect of IFN-γ was determined by incubating the supernatant obtained from YTS cells exposed to AS101 with MDCK cells for 24 hours before infecting the cells with H5N1 virus. The degree of MDCK infection was then determined 72 hours after infection using the cell infection assay described hereinabove.

As shown in FIG. 9, a significant correlation was observed between the IFN-γ levels and protection from infection.

Natural Killer (NK) Cell Killing Assay:

The effect of AS101 treatment on NK cell killing was also tested as described hereinabove.

As shown in FIG. 10A, target cell death was proportional to the ratio of NK effector cells to target cells.

As shown in FIG. 10B, increased NK killing was observed following treatment with AS101. As observed with IFN-γ secretion, high concentrations of AS101 were less effective than moderate concentrations.

In Vivo Interferon-γ (IFNγ) Assay:

The effect of AS101 on the secretion of INFγ in the lungs of H5N1 infected mice was investigated. 3 mice were treated with AS101 24 hours before infection with sub-lethal dose of H5N1 influenza. Six days after infection, the lungs were removed and homogenized with a homogenizer (Omni). INFγ levels were measured by ELISA assay six days after infection, as described hereinabove.

As shown in FIG. 11, the infection with the virus inhibits interferon production, whereas treatment with AS101 restored interferon secretion to the levels observed in uninfected mice.

PI3k-Akt Signaling:

Elevation of IFNγ levels may provide an explanation for the in vivo antiviral effects of AS101, but not for the in vitro antiviral effect of AS101 observed hereinabove.

The PI3k-Akt signaling pathway is well known to activate anti-apoptotic proteins [Li et al., Nature 1998, 396:580-584]. Influenza viruses can increase the PI3k-Akt signaling at early and middle phases of infection in order to survive [Zhirnov and Klenk, Apoptosis 2007, 12:1419-1432]. Hence, the effect of AS101 on PI3k-Akt signaling was determined in vitro in MDCK cells using a Western blot for phosphorylated Akt, as described hereinabove.

As shown in FIGS. 12A and 12B, both AS101 treatment alone and infection with H5N1 virus alone increased the level of phosphorylated Akt, whereas AS101 treatment of cells infected with H5N1 virus surprisingly resulted in dramatically reduced levels of phosphorylated Akt.

These results suggest that AS101 treatment may inhibit influenza infection via downregulation of Akt phosphorylation, in addition to the mechanism of upregulation of IFNγ levels which is effective in vivo.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A method of treating a viral infection caused by an influenza virus, the method comprising administering to a subject in need thereof a therapeutically effective amount of a tellurium-containing compound.

2. The method of claim 1, wherein said administering is initiated within 72 hours of the appearance in said subject of symptoms of said viral infection.

3. A method of prophylaxis against a viral infection caused by an influenza virus, the method comprising administering to a subject in need thereof a therapeutically effective amount of a tellurium-containing compound.

4. The method of any of claims 1 to 3, further comprising co-administering to said subject an antiviral agent.

5. Use of a tellurium-containing compound in the manufacture of a medicament for treating a viral infection caused by an influenza virus.

6. The use of claim 5, wherein said medicament is for use beginning within 72 hours of the appearance of symptoms of said viral infection.

7. Use of a tellurium-containing compound in the manufacture of a prophylactic medicament against a viral infection caused by an influenza virus.

8. The use of any of claims 5 to 7, wherein said medicament is for use in combination with an antiviral agent.

9. A tellurium-containing compound being identified for use in a method of treating a viral infection caused by an influenza virus.

10. The tellurium-containing compound of claim 9, being identified for use beginning within 72 hours of the appearance of symptoms of said viral infection.

11. A tellurium-containing compound being identified for use in a method of prophylaxis against a viral infection caused by an influenza virus.

12. The tellurium-containing compound of any of claims 9 to 11, being for use in combination with an antiviral agent.

13. A pharmaceutical composition comprising a tellurium-containing compound and a pharmaceutically acceptable carrier, the composition being identified for use in the treatment of a viral infection caused by an influenza virus.

14. A pharmaceutical composition comprising a tellurium-containing compound and a pharmaceutically acceptable carrier, the composition being identified for use in prophylaxis against a viral infection caused by an influenza virus.

15. The pharmaceutical composition of claim 13, being packaged in a packaging material, and identified in print, in or on said packaging material, for use in the treatment of said viral infection.

16. The pharmaceutical composition any of claims 13 and 15, being identified for use beginning within 72 hours of the appearance of symptoms of said viral infection.

17. The pharmaceutical composition of claim 14, being packaged in a packaging material, and identified in print, in or on said packaging material, for use in prophylaxis against said viral infection.

18. The pharmaceutical composition of any of claims 13 to 17, being identified for use in combination with an antiviral agent.

19. The pharmaceutical composition of any of claims 13 to 17, further comprising an antiviral agent.

20. A pharmaceutical composition comprising a tellurium-containing compound, an antiviral agent, and a pharmaceutically acceptable carrier.

21. A pharmaceutical kit comprising a tellurium-containing compound and an antiviral agent, each being individually packaged within the kit, the kit being identified for use in treating a viral infection caused by an influenza virus.

22. The pharmaceutical kit of claim 21, wherein said tellurium-containing compound is identified for use beginning within 72 hours of the appearance of symptoms of said viral infection.

23. A pharmaceutical kit comprising a tellurium-containing compound and an antiviral agent, each being individually packaged within the kit, the kit being identified for use in prophylaxis against a viral infection caused by an influenza virus.

24. The method, use, tellurium-containing compound, pharmaceutical kit or pharmaceutical composition of any of claims 1 to 19 and 21 to 23, wherein said influenza virus is an Influenza A virus.

25. The method, use, tellurium-containing compound, pharmaceutical kit or pharmaceutical composition of claim 24, wherein said virus is an H5N1 virus.

26. The method, use, tellurium-containing compound, pharmaceutical kit or pharmaceutical composition of claim 25, wherein said infection is an avian influenza.

27. The method, use, tellurium-containing compound, pharmaceutical kit or pharmaceutical composition of any of claims 1 to 19 and 21 to 23, wherein said virus is an H1N1 virus.

28. The method, use, tellurium-containing compound, pharmaceutical kit or pharmaceutical composition of claim 27, wherein said infection is a swine influenza.

29. The method of claim 4, the use of claim 8, the tellurium-containing compound of claim 12, the pharmaceutical composition of any of claims 19 and 20, or the pharmaceutical kit of claim 24, wherein said antiviral agent is a neuraminidase inhibitor.

30. The method, use, tellurium-containing compound, pharmaceutical kit or pharmaceutical composition of claim 29, wherein said neuraminidase inhibitor is selected from the group consisting of oseltamivir, zanamivir, laninamivir and peramivir.

31. The method, use, tellurium-containing compound, pharmaceutical kit or pharmaceutical composition of claim 30, wherein said neuraminidase inhibitor is oseltamivir.

32. The method of claim 4, the use of claim 8, the tellurium-containing compound of claim 12, the pharmaceutical composition of any of claims 19 and 20, or the pharmaceutical kit of claim 24, wherein said tellurium-containing compound and said antiviral agent act in synergy.

33. The method, use, tellurium-containing compound, pharmaceutical kit or pharmaceutical composition of any of claims 1 to 32, wherein said tellurium-containing compound comprises at least one tellurium dioxo moiety.

34. The method, use, tellurium-containing compound, pharmaceutical kit or pharmaceutical composition of claim 33, wherein said tellurium-containing compound has a general formula selected from the group consisting of:
a compound having general Formula I: 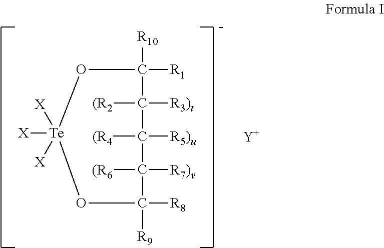
a compound having general Formula II: 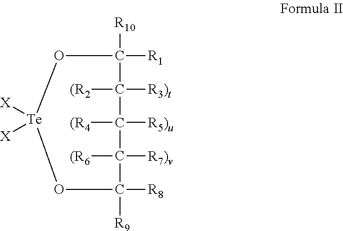
a compound having general Formula III: 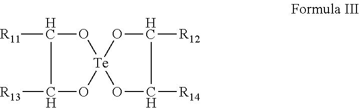
and
a compound having general Formula IV: 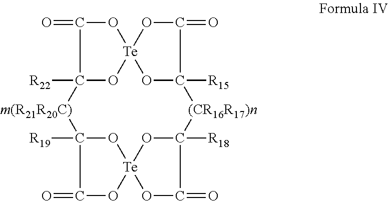
wherein:
each of t, u and v is independently 0 or 1;
each of m and n is independently 0, 1, 2 or 3;
Y is selected from the group consisting of ammonium, phsophonium, potassium, sodium and lithium;
X is a halogen atom; and
each of R₁-R₂₂ is independently selected from the group consisting of hydrogen, hydroxyalkyl, hydroxy, thiohydroxy, alkyl, alkenyl, alkynyl, alkoxy, thioalkoxy, halogen, haloalkyl, carboxy, carbonyl, alkylcarbonylalkyl, carboxyalkyl, acyl, amido, cyano, N-monoalkylamidoalkyl, N,N-dialkylamidoalkyl, cyanoalkyl, alkoxyalkyl, carbamyl, cycloalkyl, heteroalicyclic, sulfonyl, sulfinyl, sulfate, amine, aryl, heteroaryl, phosphate, phosphonate and sulfonamido.

35. The method, use, tellurium-containing compound, pharmaceutical kit or pharmaceutical composition of claim 34, wherein said tellurium-containing compound has said general Formula I.

36. The method, use, tellurium-containing compound, pharmaceutical kit or pharmaceutical composition of claim 35, wherein t, u and v are each 0.

37. The method, use, tellurium-containing compound, pharmaceutical kit or pharmaceutical composition of claim 36, wherein each of R₁, R₈, R₉ and R₁₀ is hydrogen.

38. The method, use, tellurium-containing compound, pharmaceutical kit or pharmaceutical composition of claim 37, wherein X is chloro.

39. The method, use, tellurium-containing compound, pharmaceutical kit or pharmaceutical composition of claim 38, wherein Y is ammonium.

40. The method, use, tellurium-containing compound, pharmaceutical kit or pharmaceutical composition of any of claims 1 to 32, wherein said tellurium-containing compound is ammonium trichloro(dioxoethylene-O,O′)tellurate (AS101).

41. The method, use, tellurium-containing compound, pharmaceutical kit or pharmaceutical composition of claim 34, wherein said compound has said general Formula IV.

42. The method, use, tellurium-containing compound, pharmaceutical kit or pharmaceutical composition of claim 41, wherein each of m and n is 0.

43. The method, use, tellurium-containing compound, pharmaceutical kit or pharmaceutical composition of claim 42, wherein each of R₁₅, R₁₈, R₁₉ and R₂₂ is hydrogen.

44. The method, use, tellurium-containing compound, pharmaceutical kit or pharmaceutical composition of any of claims 1 to 32, wherein said tellurium-containing compound is SAS.
